(19) United States
(12) Patent Application Publication
(10) Pub. No.: US 2022/0226377 A1
(43) Pub. Date: Jul. 21, 2022

(54) TREATMENT OF DIABETES USING IMMUNE CELLS REPROGRAMMED EX VIVO BY REGENERATIVE CELLS

(71) Applicant: CREATIVE MEDICAL TECHNOLOGIES, INC., PHOENIX, AZ (US)

(72) Inventors: Thomas Ichim, San Diego, CA (US); Amit Patel, Salt Lake City, UT (US)

(73) Assignee: CREATIVE MEDICAL TECHNOLOGIES, INC., PHOENIX, AZ (US)

(21) Appl. No.: 17/578,298

(22) Filed: Jan. 18, 2022

Related U.S. Application Data
(60) Provisional application No. 63/138,776, filed on Jan. 18, 2021.

(51) Int. Cl. A61K 35/17; C12N 5/0775; C12N 5/073; A61K 35/28

(52) U.S. Cl. CPC A61K 35/17; C12N 5/0665; C12N 2501/59; A61K 35/28; C12N 5/0605

(57) ABSTRACT

Disclosed are methods of ameliorating, inhibition, and/or reversing diabetes utilizing immune cells that have been reprogrammed ex vivo by contact with regenerative cells. In one embodiment said reprogrammed immune cells comprise peripheral blood mononuclear cells obtained from the patient in need of treatment wherein said cells are endowed with properties of immune modulation, and/or suppression of inflammation, and/or restoration of insulin sensitivity, and/or pancreatic regeneration. In one embodiment regenerative cells used for reprogramming are mesenchymal stem cells. In one particular embodiment said cells are umbilical cord derived mesenchymal stem cells. Culture of peripheral blood mononuclear cells together with said regenerative cells is performed in the presence of interleukin-2 and/or an mTOR inhibitor. In one embodiment said mTOR inhibitor comprises rapamycin and/or a derivative thereof.

### CROSS-REFERENCE TO RELATED APPLICATIONS

This application claims priority to U.S. Provisional Application No. 63/138,776, filed Jan. 18, 2021, which is herein incorporated by reference in its entirety.

### FIELD OF THE INVENTION

The invention pertains to the field of diabetes, more specifically the invention relates to treatment of diabetes with immune modulatory cells, more specifically, the invention relates to treatment of heart failure with ex vivo stem cell reprogrammed cells.

### BACKGROUND

There are two main forms of diabetes: Type 1 diabetes and Type 2. In Type 1 diabetes, also known as insulin-dependent diabetes mellitus (IDDM), or juvenile diabetes, the patient's pancreas produces little or no insulin, believed to be in part the result of autoimmune attached on the insulin producing beta-cells in the pancreas. It's one of the most costly, chronic diseases of childhood and one you never outgrow. It is believed that more than one million Americans have IDDM. Patients with full-blown IDDM must take multiple insulin injections daily or continually infuse insulin through a pump, and test their blood sugar by pricking their fingers for blood six or more times per day. Neither dietary therapy nor treatment with an oral hypoglycemic agent is effective, and only treatment with insulin is effective. Ketonemia and acidosis due to the loss of insulin secreting capacity, and if untreated, may result in diabetic coma. Because numerous factors such as stress, hormones, growth, physical activity, medications, illness/infection, and fatigue effect insulin utilization, even a strictly monitored program of insulin administration does not mimic the endogenous functions of the pancreas, and as a result numerous complications develop. Non-Insulin Dependent Diabetes Mellitus (NIDDM), or adult-onset diabetes, is associated with impairment of peripheral tissue response to insulin. NIDDM is believed to afflict approximately 18.2 million people in the US and as a result of the obesity epidemic, substantially younger patients are beginning to be diagnosed with this condition. The economic burden of NIDDM is witnessed in statistics demonstrating that on average, the health care costs for NIDDM patients are expensive. Insulin resistance is present in almost all obese individuals W. However, compensatory insulin production by beta-cells usually occurs, thus preventing hyperglycemia. In response to prolonged insulin resistance, as well as other factors, beta cell insulin production eventually lose ability to cope with the increasing insulin demands and postprandial hyperglycemia occurs, characterizing the transition between normal glucose tolerance and abnormal glucose tolerance. Subsequently, the liver starts secreting glucose through hepatic gluconeogenesis (generation of glucose from substrates that are not sugars, not from glycogen) and hyperglycemia is observed even in the fasting state. In contrast to IDDM, NIDDM presents only a small degree of ketonemia and acidosis although the insulin action is reduced from normal, and treatment with insulin is not always required. The greatest clinical challenge in this disease is the prevention of the long-term complications, many of which involve vascular, ocular and renal systems. Although various agents are utilized to increase glucose sensitivity, or to stimulate insulin secretion, these approaches are not optimal because they do not exactly mimic the physiological control of post-prandial insulin secretion. Accordingly, the fluctuations of glucose, as well as downstream metabolic consequences end up causing macrovascular pathology such as coronary atherosclerosis, and increased risk of stroke, as well as microvascular pathology such as macular degeneration and renal failure. Additionally, neuropathies are often present associated with hyperglycemia. There are numerous treatments available for NIDDM; these depend on patient-specific characteristics, as well as severity of disease. The treatment goal in diabetes treatment is to bring plasma glucose levels down to as near normal levels, for example 80-120 milligrams per deciliter (mg/dl) before meals and 100-140 mg/dl at night. Numerous medical tests are known in the art for monitoring glucose, as well as cholesterol and lipid levels. The goal of maintaining normal glucose levels is judged in some ways by the ability to prevent secondary complications, such as retinopathy, neuropathy, vascular disease, and strokes.

### SUMMARY

Preferred embodiments herein are directed to methods of treatment of diabetes comprising the steps of: a) identifying a patient suffering from diabetes; b) withdrawing an immunologically relevant cellular population from said patient; c) contacting said immunologically relevant cellular population from said patient with one or more regenerative cells from another individual for a sufficient period of time to endow onto said immunologically relevant cells properties that are therapeutically relevant to diabetes; and d) administering said reprogrammed immunologically relevant cells to a patient in need of treatment.

Preferred methods include embodiments, wherein said patient suffering from diabetes suffers from type 1 diabetes.

Preferred methods include embodiments, wherein said patient suffering from diabetes suffers from type 2 diabetes.

Preferred methods include embodiments, wherein said immunologically relevant cellular population comprises peripheral blood mononuclear cells.

Preferred methods include embodiments, wherein said immunologically relevant cellular population is cultured together with said regenerative population in the presence of a tolerogenic adjuvant.

Preferred methods include embodiments, wherein said tolerogenic adjuvant is an agent endowing immune regulatory properties to said immunologically relevant cells.

Preferred methods include embodiments, wherein said immune regulatory properties are suppression of T cell proliferation in response to T cell receptor ligation.

Preferred methods include embodiments, wherein said immune regulatory properties are suppression of T cell cytokine production in response to T cell receptor ligation.

Preferred methods include embodiments, wherein said cytokine is interleukin-1.

Preferred methods include embodiments, wherein said cytokine is interleukin-3.

Preferred methods include embodiments, wherein said cytokine is interleukin-7.

Preferred methods include embodiments, wherein said cytokine is interleukin-9.

Preferred methods include embodiments, wherein said cytokine is interleukin-12.

Preferred methods include embodiments, wherein said cytokine is interleukin-15.

Preferred methods include embodiments, wherein said cytokine is interleukin-18.

Preferred methods include embodiments, wherein said cytokine is interleukin-22.

Preferred methods include embodiments, wherein said cytokine is interleukin-23.

Preferred methods include embodiments, wherein said cytokine is interleukin-27.

Preferred methods include embodiments, wherein said cytokine is interleukin-33.

Preferred methods include embodiments, wherein said cytokine is interferon alpha.

Preferred methods include embodiments, wherein said cytokine is interferon beta.

Preferred methods include embodiments, wherein said cytokine is interferon gamma.

Preferred methods include embodiments, wherein said cytokine is interferon tau.

Preferred methods include embodiments, wherein said cytokine is interferon omega.

Preferred methods include embodiments, wherein said immune regulatory properties are suppression of T cell cytotoxic activity in response to T cell receptor ligation.

Preferred methods include embodiments, wherein said immune regulatory properties are suppression of dendritic cell maturing activity in response to T cell receptor ligation.

Preferred methods include embodiments, wherein said dendritic cell maturation comprises reduction of dendritic cell phagocytic activity.

Preferred methods include embodiments, wherein said dendritic cell maturation comprises reduction of dendritic cell production of PD-L1.

Preferred methods include embodiments, wherein said dendritic cell maturation comprises reduction of dendritic cell secretion of interleukin 10.

Preferred methods include embodiments, wherein said dendritic cell maturation comprises reduction of dendritic cell production of TGF-beta.

Preferred methods include embodiments, wherein said dendritic cell maturation comprises reduction of dendritic cell production of indolamine 2,3 deoxygenase.

Preferred methods include embodiments, wherein said dendritic cell maturation comprises reduction of dendritic cell expression of Fas Ligand.

Preferred methods include embodiments, wherein said dendritic cell maturation comprises reduction of dendritic cell expression of HLA-G.

Preferred methods include embodiments, wherein said dendritic cell maturation comprises reduction of dendritic cell expression of arginase activity.

Preferred methods include embodiments, wherein said dendritic cell maturation comprises reduction of dendritic cell expression of TLR-2.

Preferred methods include embodiments, wherein said dendritic cell maturation comprises reduction of dendritic cell expression of TLR-5.

Preferred methods include embodiments, wherein said dendritic cell maturation comprises reduction of dendritic cell expression of interleukin 1 receptor antagonist.

Preferred methods include embodiments, wherein said dendritic cell maturation comprises reduction of dendritic cell expression of ILT3.

Preferred methods include embodiments, wherein said dendritic cell maturation comprises reduction of dendritic cell expression of LAP.

Preferred methods include embodiments, wherein said tolerogenic adjuvant is an inhibitor of NF-kappa B.

Preferred methods include embodiments, wherein said inhibitor of NF-kappa B is aspirin.

Preferred methods include embodiments, wherein said inhibitor of NF-kappa B is interleukin-4.

Preferred methods include embodiments, wherein said inhibitor of NF-kappa B is vitamin D3.

Preferred methods include embodiments, wherein said inhibitor of NF-kappa B is resveratrol

Preferred methods include embodiments, wherein said inhibitor of NF-kappa B is pterostilbene.

Preferred methods include embodiments, wherein said inhibitor of NF-kappa B is fish oil.

Preferred methods include embodiments, wherein said inhibitor of NF-kappa B is nattokinase.

Preferred methods include embodiments, wherein said inhibitor of NF-kappa B is sulforaphane.

Preferred methods include embodiments, wherein said inhibitor of NF-kappa B is melatonin.

Preferred methods include embodiments, wherein said inhibitor of NF-kappa B is ascorbic acid.

Preferred methods include embodiments, wherein said inhibitor of NF-kappa B is deohydroascorbic acid.

Preferred methods include embodiments, wherein said inhibitor of NF-kappa B is zinc.

Preferred methods include embodiments, wherein said inhibitor of NF-kappa B is trivalent cadmium.

Preferred methods include embodiments, wherein said inhibitor of NF-kappa B is carbon 60.

Preferred methods include embodiments, wherein said inhibitor of NF-kappa B is EDTA.

Preferred methods include embodiments, wherein said inhibitor of NF-kappa B is TGF-beta.

Preferred methods include embodiments, wherein said inhibitor of NF-kappa B is interleukin-10.

Preferred methods include embodiments, wherein said inhibitor of NF-kappa B is interleukin-20.

Preferred methods include embodiments, wherein said inhibitor of NF-kappa B is VEGF.

Preferred methods include embodiments, wherein said inhibitor of NF-kappa B is EGF.

Preferred methods include embodiments, wherein said inhibitor of NF-kappa B is NGF.

Preferred methods include embodiments, wherein said inhibitor of NF-kappa B is BDNF.

Preferred methods include embodiments, wherein said inhibitor of NF-kappa B is GDNF.

Preferred methods include embodiments, wherein said inhibitor of NF-kappa B is astralagous extract.

Preferred methods include embodiments, wherein said inhibitor of NF-kappa B is a gingingoside.

Preferred methods include embodiments, wherein said inhibitor of NF-kappa B is retinol.

Preferred methods include embodiments, wherein said inhibitor of NF-kappa B is ikappa B.

Preferred methods include embodiments, wherein said inhibitor of NF-kappa B is frankincense.

Preferred methods include embodiments, wherein said inhibitor of NF-kappa B is sheep sorrel extract.

Preferred methods include embodiments, wherein said inhibitor of NF-kappa B is acetaminophen.

Preferred methods include embodiments, wherein said immunologically relevant cells are T cells.

Preferred methods include embodiments, wherein wherein said T cells are CD4 cells.

Preferred methods include embodiments, wherein wherein said T cells are CD8 cells.

Preferred methods include embodiments, wherein said T cells are NKT cells.

Preferred methods include embodiments, wherein said T cells are gamma delta T cells.

Preferred methods include embodiments, wherein said T cells are Th1 cells.

Preferred methods include embodiments, wherein said Th1 cells have a propensity for producing interferon gamma over interleukin 4 upon stimulation via the CD3 protein.

Preferred methods include embodiments, wherein said Th1 cells express STAT4.

Preferred methods include embodiments, wherein said Th1 cells express STAT1.

Preferred methods include embodiments, wherein said Th1 cells express T-bet.

Preferred methods include embodiments, wherein said Th1 cells express CCR1.

Preferred methods include embodiments, wherein said Th1 cells express CCR5.

Preferred methods include embodiments, wherein said Th1 cells express CXCR3.

Preferred methods include embodiments, wherein said Th1 cells express CD119.

Preferred methods include embodiments, wherein said Th1 cells express interferon gamma receptor II.

Preferred methods include embodiments, wherein said Th1 cells express IL-18 receptor.

Preferred methods include embodiments, wherein said Th1 cells express IL-12 receptor.

Preferred methods include embodiments, wherein said Th1 cells express IL-27 receptor.

Preferred methods include embodiments, wherein said T cells are Th2 cells.

Preferred methods include embodiments, wherein said Th2 cells have a proclivity to produce more interleukin-4 than interferon gamma upon stimulation via CD3.

Preferred methods include embodiments, wherein said Th2 cells express GATA-3.

Preferred methods include embodiments, wherein said Th2 cells express IRF-4.

Preferred methods include embodiments, wherein said Th2 cells express STAT5.

Preferred methods include embodiments, wherein said Th2 cells express STAT6.

Preferred methods include embodiments, wherein said Th2 cells express CCR3.

Preferred methods include embodiments, wherein said Th2 cells express CCR4.

Preferred methods include embodiments, wherein said Th2 cells express CCR8.

Preferred methods include embodiments, wherein said Th2 cells express CXCR4.

Preferred methods include embodiments, wherein said Th2 cells express interleukin-4 receptor.

Preferred methods include embodiments, wherein said Th2 cells express interleukin-33 receptor.

Preferred methods include embodiments, wherein said T cells are Th9 cells.

Preferred methods include embodiments, wherein said Th9 cell produces interleukin-9.

Preferred methods include embodiments, wherein said Th9 cell expresses IRF4.

Preferred methods include embodiments, wherein said Th9 cell expresses PU.1.

Preferred methods include embodiments, wherein said Th9 cell secretes CCL17.

Preferred methods include embodiments, wherein said Th9 cell secretes CCL22.

Preferred methods include embodiments, wherein said Th9 cell secretes IL-10.

Preferred methods include embodiments, wherein said Th9 cell expresses TGF-beta receptor II.

Preferred methods include embodiments, wherein said T cell is a follicular helper T cell.

Preferred methods include embodiments, wherein said follicular helper T cell expresses bcl-6.

Preferred methods include embodiments, wherein said follicular helper T cell expresses c-maf.

Preferred methods include embodiments, wherein said follicular helper T cell expresses stat-3.

Preferred methods include embodiments, wherein said follicular helper T cell secretes CXCL-13.

Preferred methods include embodiments, wherein said follicular helper T cell secretes interferon gamma.

Preferred methods include embodiments, wherein said follicular helper T cell secretes interleukin-4.

Preferred methods include embodiments, wherein said follicular helper T cell secretes IL-10.

Preferred methods include embodiments, wherein said follicular helper T cell secretes IL-17A.

Preferred methods include embodiments, wherein said follicular helper T cell secretes IL-17F.

Preferred methods include embodiments, wherein said follicular helper T cell secretes IL-21.

Preferred methods include embodiments, wherein said follicular helper T cell expresses BTLA-4.

Preferred methods include embodiments, wherein said follicular helper T cell secretes CD40 ligand.

Preferred methods include embodiments, wherein said follicular helper T cell expresses CD57.

Preferred methods include embodiments, wherein said follicular helper T cell expresses CD84.

Preferred methods include embodiments, wherein said follicular helper T cell expresses CXCR-4.

Preferred methods include embodiments, wherein said follicular helper T cell expresses CXCR-5.

Preferred methods include embodiments, wherein said follicular helper T cell expresses ICOS.

Preferred methods include embodiments, wherein said follicular helper T cell expresses IL-6 receptor.

Preferred methods include embodiments, wherein said follicular helper T cell expresses IL-21 receptor.

Preferred methods include embodiments, wherein said follicular helper T cell expresses CD10.

Preferred methods include embodiments, wherein said follicular helper T cell expresses OX40.

Preferred methods include embodiments, wherein said follicular helper T cell expresses PD-1.

Preferred methods include embodiments, wherein said follicular helper T cell expresses CD150.

Preferred methods include embodiments, wherein said T cell is a Th17 cell.

Preferred methods include embodiments, wherein said Th17 cell secretes interleukin-17A.

Preferred methods include embodiments, wherein said Th17 cell secretes interleukin-17F.

Preferred methods include embodiments, wherein said Th17 cell secretes IL-21.

Preferred methods include embodiments, wherein said Th17 cell secretes IL-26.

Preferred methods include embodiments, wherein said Th17 cell secretes CCL20.

Preferred methods include embodiments, wherein said Th17 cell expresses BATF.

Preferred methods include embodiments, wherein said Th17 cell expresses IRF4.

Preferred methods include embodiments, wherein said Th17 cell expresses ROR alpha.

Preferred methods include embodiments, wherein said Th17 cell expresses ROR gamma.

Preferred methods include embodiments, wherein said Th17 cell expresses STAT3.

Preferred methods include embodiments, wherein said Th17 cell expresses CCR4.

Preferred methods include embodiments, wherein said Th17 cell expresses CCR6.

Preferred methods include embodiments, wherein said Th17 cell expresses IL-1 receptor.

Preferred methods include embodiments, wherein said Th17 cell expresses IL-6 receptor alpha.

Preferred methods include embodiments, wherein said Th17 cell expresses IL-21 receptor.

Preferred methods include embodiments, wherein said Th17 cell expresses IL-23 receptor.

### BRIEF DESCRIPTION OF THE DRAWINGS

FIG. 1 is a bar graph showing the percentage of diabetes in mice that were untreated, treated with STZ, treated with STZ+untrained T cells, or STZ+trained T cells (ImmCelz).

FIG. 2 is a bar graph showing VEGF levels of mice that were untreated, treated with STZ, treated with STZ+untrained T cells or STZ+trained T cells (ImmCelz).

FIG. 3 is a bar graph showing EGF levels of mice that were untreated, treated with STZ, treated with STZ+untrained T cells or STZ+trained T cells (ImmCelz).

FIG. 4 is a bar graph showing IGF levels of mice that were untreated, treated with STZ, treated with STZ+untrained T cells or STZ+trained T cells (ImmCelz).

FIG. 5 is a bar graph showing HGF levels of mice that were untreated, treated with STZ, treated with STZ+untrained T cells or STZ+trained T cells (ImmCelz).

### DETAILED DESCRIPTION OF THE INVENTION

In one embodiment of the invention regeneration of pancreatic tissue is induced by administration of autologous peripheral blood mononuclear cells that have been cultured with regenerative cells. In one embodiment said regenerative cells are umbilical cord mesenchymal stem cells. In one embodiment cells are cultured at a ratio of 1 peripheral blood mononuclear cell to one umbilical cord mesenchymal stem cell.

IMMCELZ™ are a proprietary type of treated T regulatory cell have regenerative properties, the disclosure of which is described in detail in U.S. Provisional Application No. 63/270,678, which was filed Oct. 22, 2021, and is hereby incorporated by reference in its entirety.

Throughout this specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the invention, for example. Some embodiments may consist of or consist essentially of one or more elements, method steps, and/or methods of the invention. It is contemplated that any method or composition described herein can be implemented with respect to any other method or composition described herein. The scope of the present application is not intended to be limited to the particular embodiments of the process, machine, manufacture, composition of matter, means, methods and steps described in the specification.

Throughout this specification, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe, etc. A second exemplary method of administering is by a direct mechanism such as, local tissue administration, oral ingestion, transdermal patch, topical, inhalation, suppository, etc.

The term “allogeneic,” as used herein, refers to cells of the same species that differ genetically from cells of a host.

The term “autologous,” as used herein, refers to cells derived from the same subject. The term “engraft” as used herein refers to the process of stem cell incorporation into a tissue of interest in vivo through contact with existing cells of the tissue.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

As used herein, the term “activated fibroblasts” refers to fibroblasts treated with one or more agents and/or stimuli capable of inducing one or more alterations in the cell: metabolic, immunological, growth factor-secreting, surface marker expression, and/or production of microvesicles. Examples of agents include epidermal growth factor (EGF; (Peprotech), Transforming Growth Factor-alpha (TGF-alpha; Peprotech), basic Fibroblast Growth Factor (bFGF; Peprotech), brain-derived neurotrophic factor (BDNF; R&D Systems), and Keratinocyte Growth Factor (KGF; Peprotech). EGF is a potent mitogenic factor for a variety of cultured ectodermal and mesodermal cells and has a profound effect on the differentiation of specific cells in vivo and in vitro and of some fibroblasts in cell culture. The EGF precursor exists as a membrane-bound molecule which is proteolytically cleaved to generate the 53-amino acid peptide hormone that stimulates cells. A preferred mitogenic growth factor is EGF. EGF is preferably added to the basal culture medium at a concentration of between 5 and 500 ng/ml or of at least 5 and not higher than 500 ng/ml. A preferred concentration is at least 10, 20, 25, 30, 40, 45, or 50 ng/ml and not higher than 500, 450, 400, 350, 300, 250, 200, 150, or 100 ng/ml. A more preferred concentration is at least 50 and not higher than 100 ng/ml. An even more preferred concentration is about 50 ng/ml or 50 ng/ml. The same concentrations could be used for a FGF, preferably for FGF10 or FGF7. If more than one FGF is used, for example, FGF7 and FGF10, the concentration of a FGF is as defined above and refers to the total concentration of FGF used. During culturing of stem cells, the mitogenic growth factor is preferably added to the culture medium every second day, while the culture medium is refreshed preferably every fourth day. Any member of the bFGF family may be used. In some cases, FGF7 and/or FGF10 is used. FGF7 is also known as KGF (Keratinocyte Growth Factor).

“Cell culture” is an artificial in vitro system containing viable cells, whether quiescent, senescent or (actively) dividing. In a cell culture, cells are grown and maintained at an appropriate temperature, typically a temperature of 37.degree. C. and under an atmosphere typically containing oxygen and CO.sub.2. Culture conditions may vary widely for each cell type though, and variation of conditions for a particular cell type can result in different phenotypes being expressed. The most commonly varied factor in culture systems is the growth medium. Growth media can vary in concentration of one or more of nutrients, growth factors, and the presence of other components. The growth factors used to supplement media are often derived from animal blood, such as calf serum.

As used herein, the term “conditioned medium of fibroblast regenerative cells” refers to a liquid media that has been in contact with cells, wherein the cells produce one or more factors that enter the media, thus bestowing upon the media at least one therapeutic activity.

The term “individual”, as used herein, refers to a human or animal that may or may not be housed in a medical facility and may be treated as an outpatient of a medical facility. The individual may or may not be receiving one or more medical compositions from a medical practitioner and/or via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method and/or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “prevent” or “preventing” refers to a method wherein a medical condition or onset of at least one symptom thereof is kept from occurring.

The term “subject” or “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to promote formation of new blood vessels and associated vasculature (angiogenesis) and/or an amount sufficient to promote repair or remodeling of existing blood vessels and associated vasculature. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

The invention, in some embodiments, teaches the application of Immunological tolerance to the condition of alloantigen reactivity and autoimmunity. In one embodiment the invention teaches the treatment of diabetes. In one embodiment the invention teaches the treatment and/or reversion of type 1 diabetes. It is known that a cardinal feature of the immune system, is allowing for recognition and elimination of pathological threats, while selectively ignoring antigens that belong to the body. Traditionally, autoimmune conditions such as type 1 diabetes or conditions associated with cytokine storm, or allograft rejection are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia.

The invention provides novel stem cell types, methods of manufacture, and therapeutic uses. Provided are means of deriving stem cells possessing regenerative, immune modulatory, anti-inflammatory, and angiogenic/neurogenic activity from umbilical cord tissue such as Wharton's Jelly. In some embodiments manipulation of stem cell “potency” is disclosed through hypoxic manipulation, growth on non-xenogeneic conditions, as well as addition of epigenetic modulators.

The cells of the invention are cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, peripheral blood, hair follicle, cord blood, Wharton's Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In some embodiments, the population of cells, the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell.

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

In some embodiments, a population of umbilical cord cells is optionally obtained, the population is then optionally contacted via culturing with a population of progenitor for T regulatory cells, wherein the culturing conditions allow for the generation of T regulatory cells, then the generated T regulatory cells are administered to an individual.

In another embodiment of the invention, biologically useful immune cells are generated after culture with regenerative cells, and/or stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention, administration of cells of the invention is performed for suppression of an inflammatory and/or autoimmune disease. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric.acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

### EXAMPLES

Treatment of Type 1 Diabetes Model

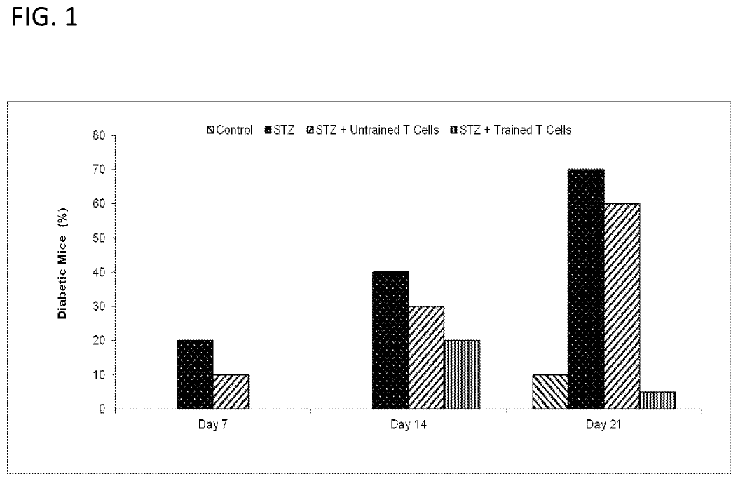

Autoimmune diabetes, similar to juvenile diabetes (type 1 diabetes) was treated with cellular therapy. Female non obese diabetic (NOD) mice where induced to undergo hyperaccelerated diabetes by administration of streptozotocin (STZ). Mice (10 per group) were untreated, treated with STZ, treated with STZ+untrained T cells or STZ+trained T cells (ImmCelz). Diabetes onset was determined by blood glucose levels. Inhibition of diabetes onset was observed. Results are shown in FIG. 1.

Induction of Regenerative Cytokines Type 1 Diabetes Model

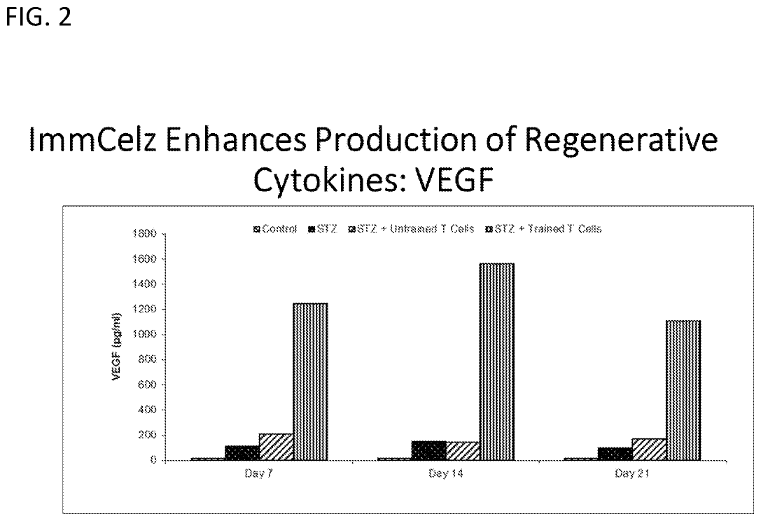

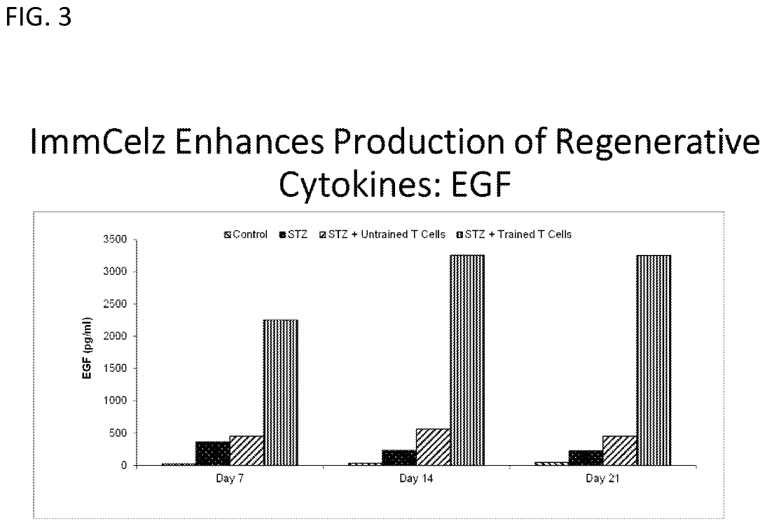

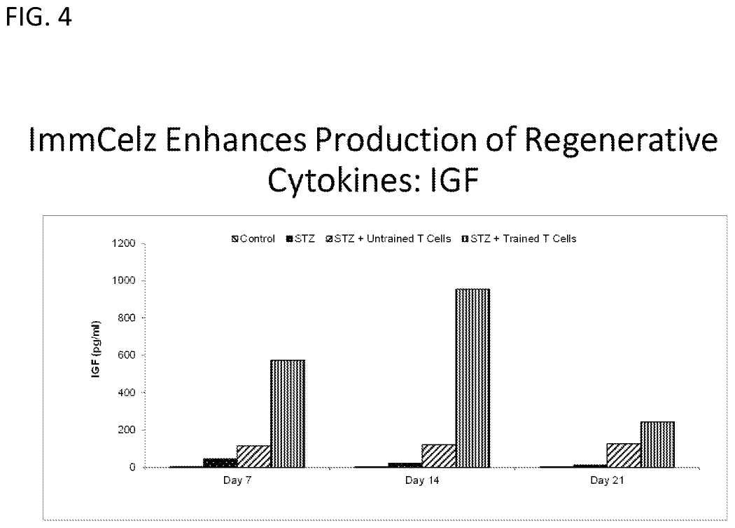

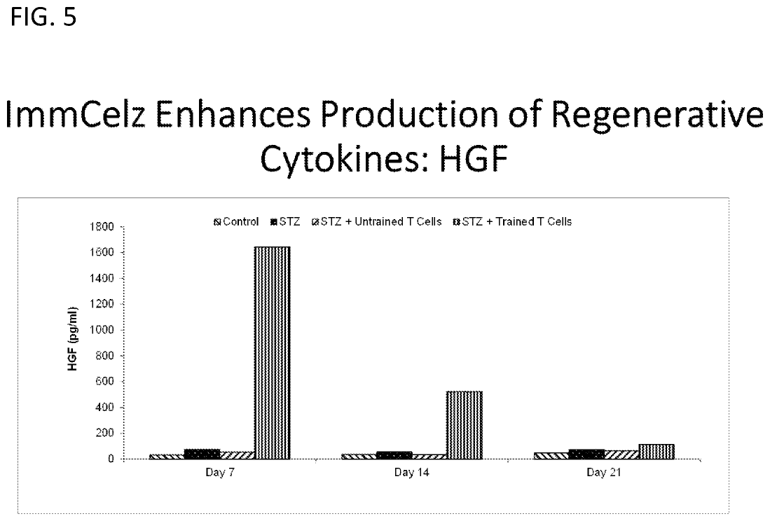

Autoimmune diabetes, similar to juvenile diabetes (type 1 diabetes) was treated with cellular therapy. Female non obese diabetic (NOD) mice where induced to undergo hyperaccelerated diabetes by administration of streptozotocin (STZ). Mice (10 per group) were untreated, treated with STZ, treated with STZ+untrained T cells or STZ+trained T cells (ImmCelz). Cytokines where analyzed by ELISA from tail vein blood draw. Results are shown in FIGS. 2-5.

1. A method of treatment of diabetes comprising the steps of: a) identifying a patient suffering from diabetes; b) withdrawing an immunologically relevant cellular population from said patient; c) contacting said immunologically relevant cellular population from said patient with one or more regenerative cells from another individual for a sufficient period of time to endow onto said immunologically relevant cells properties that are therapeutically relevant to diabetes; and d) administering said reprogrammed immunologically relevant cells to a patient in need of treatment

2. The method of claim 1, wherein said contacting of said immunologically relevant cellular population with said regenerative cells is performed by culture of conditioned media from said regenerative cell with said immunologically relevant cell.

3. The method of claim 2, wherein said immunologically relevant cell comprises peripheral blood mononuclear cells derived from the patient in need of treatment.

4. The method of claim 2, wherein said immunologically relevant cell is a CD4 T cell.

5. The method of claim 2, wherein said immunologically relevant cell is a CD8 T cell.

6. The method of claim 2, wherein said immunologically relevant cell is a gamma delta T cell.

7. The method of claim 1, wherein said regenerative cell is a mesenchymal stem cell.

8. The method of claim 7, wherein said mesenchymal stem cell is derived from the umbilical cord.

9. The method of claim 8, wherein said umbilical cord derived mesenchymal stem cells is a JadiCell.

10. The method of claim 9, wherein said Jadicell is activated before collection of culture media for which said culture media will be incubated with said immunologically relevant cell.

11. The method of claim 10, wherein said activation of said JadiCell is performed by treatment with an inflammatory cytokine.

12. The method of claim 11, wherein said inflammatory cytokine is selected from a group comprising of: interferon gamma, tnf-alpha, interleukin-18, and interleukin-33.

13. The method of claim 12, wherein said inflammatory cytokine is interferon gamma.

14. The method of claim 13, wherein culture of Jadicells comprises contact with 1-1000 internationally units of interferon gamma for a period of time of 1 second to 3 months.

15. The method of claim 14, wherein said Jadicells are cultured in interferon gamma at a concentration of 100 international units per ml for a period of time of 48 hours.

16. The method of claim 1, wherein said immunologically relevant cells are cultured in the presence of conditioned media from said regenerative cells and a T cell mitogen is added.

17. The method of claim 16 wherein said T cell mitogen is selected from a group comprising of: IL-2, anti-CD3, IL-7 and IL-12.

18. The method of claim 1, wherein subsequent to culture of immunologically relevant cells with said regenerative cells, that T regulatory cells are isolated and administered into a patient in need of treatment.

19. The method of claim 18, wherein said T regulatory cells possess FoxP3.

20. The method of claim 18, wherein said T regulatory cells are isolated by selection for CD4 and CD25.
